(19) United States
(12) Patent Application Publication
(10) Pub. No.: US 2026/0191752 A1
(43) Pub. Date: Jul. 9, 2026

(54) COMPOSITION SUITABLE FOR KERATIN FIBERS

(71) Applicant: L'OREAL, Paris (FR)

(72) Inventors: Ayano TAKEDA, Kawasaki-shi (JP); Nicholas MARET, Kawasaki-shi (JP); Natsumi KOMURE, Kawasaki-shi (JP); Adelino Kaoru NAKANO, Kawasaki-shi (JP)

(21) Appl. No.: 19/135,697

(22) PCT Filed: Jul. 20, 2023
(86) PCT No.: PCT/JP2023/027472

§ 371 Date: Jun. 4, 2025

(30) Foreign Application Priority Data
Dec. 9, 2022 (JP) 2022-197247
Jan. 18, 2023 (FR) 2300474

(51) Int. Cl. A61K 8/365; A45D 7/06; A61K 8/44; A61Q 5/06

(52) U.S. Cl. CPC A61K 8/365; A45D 7/06; A61K 8/44; A61Q 5/06; A45D 2200/25

(57) ABSTRACT

The present invention mainly relates to a composition for keratin fibers, such as hair, comprising: (a) at least one compound selected from the group consisting of glyoxylic acid, glyoxylic acid solvates, glyoxylic acid salts, glyoxylic acid esters, glyoxylic acid amides, and mixtures thereof; and (b) at least one compound, different from the (a) compound, selected from the group consisting of keto acids, salts thereof, and mixtures thereof, wherein the amount of the (a) compound(s) in the composition is less than 15% by weight relative to the total weight of the composition, and the amount of the (b) compound(s) in the composition is 10% by weight or less. The present invention can provide keratin fibers such as hair with good texture, strength, and resistance to humidity which can lengthen reshaping effects.

### TECHNICAL FIELD

The present invention mainly relates to a composition which is suitable for reshaping, preferably straightening, keratin fibers such as hair.

### BACKGROUND ART

Straightening keratin fibers such as hair by heating the keratin fibers with an iron is popular for making unruly keratin fibers manageable and making keratin fibers have less volume and look more desirable. Such a straightening method using a heating iron is convenient for quick styling, but the style lasts only a short period of time, e.g., until the next shampooing. A long-lasting straightening method is required for many consumers.

On the other hand, conventional permanent straightening methods using a reducing agent and an alkaline agent, as well as an oxidizing agent, with heating, can provide keratin fibers with a long-lasting style, but they have several drawbacks such as a long processing time, damage to the keratin fibers, and malodor.

WO 2014/131469 discloses a process for straightening keratin fibers by heating with an iron, without using reducing and oxidizing agents, wherein glyoxylic acid is used to straighten the keratin fibers.

### DISCLOSURE OF INVENTION

There is still a need to improve the reshaping of keratin fibers based on treating the keratin fibers with glyoxylic acid or a derivative thereof.

An objective of the present invention is to provide a composition which is useful for reshaping keratin fibers such as hair, based on treating the keratin fibers with glyoxylic acid or a derivative thereof, and which can provide the keratin fibers with good texture such as moisturizing feeling, strength, and resistance to humidity which can lengthen the reshaping effects.

The above objective of the present invention can be achieved by a composition for keratin fibers, such as hair, comprising:
(a) at least one compound selected from the group consisting of glyoxylic acid, glyoxylic acid solvates, glyoxylic acid salts, glyoxylic acid esters, glyoxylic acid amides, and mixtures thereof; and
(b) at least one compound, different from the (a) compound, selected from the group consisting of keto acids, salts thereof, and mixtures thereof,
wherein
the amount of the (a) compound(s) in the composition is less than 15% by weight relative to the total weight of the composition, and
the amount of the (b) compound(s) in the composition is 10% by weight or less.

The amount of the (a) compound(s) in the composition according to the present invention may be 0.1% by weight or more, preferably 0.5% by weight or more, and more preferably 1% by weight or more, relative to the total weight of the composition.

The (b) compound may be selected from levulinic acid, a salt thereof, and a mixture thereof.

The amount of the (b) compound in the composition according to the present invention may be 0.1% by weight or more, preferably 0.5% by weight or more, and more preferably 1% by weight or more, relative to the total weight of the composition.

The composition according to the present invention may further comprise (c) water.

The amount of the (c) water in the composition according to the present invention may be from 20% to 95% by weight, preferably from 30% to 90% by weight, and more preferably from 40% to 85% by weight, relative to the total weight of the composition.

The composition according to the present invention may further comprise (d) at least one betaine compound.

The (d) betaine compound may be selected from the group consisting of trimethylglycine, carnitine, and L-proline betaine or stachydrine, and mixtures thereof.

The amount of the (d) betaine compound(s) in the composition according to the present invention may be from 0.01% to 15% by weight, preferably from 0.05% to 10% by weight, and more preferably from 0.1% to 5% by weight, relative to the total weight of the composition.

The pH of the composition according to the present invention may be 7 or less, preferably 6 or less, and more preferably 5 or less.

The composition according to the present invention may not comprise any ammonia or thiol compound, or may comprise less than 1% by weight, preferably less than 0.5% by weight, and more preferably less than 0.1% by weight of ammonia or thiol compound, relative to the total weight of the composition.

The composition according to the present invention may not comprise any reducing agent or oxidizing agent, or may comprise less than 1% by weight, preferably less than 0.5% by weight, and more preferably less than 0.1% by weight of a reducing agent or an oxidizing agent, relative to the total weight of the composition.

The composition according to the present invention may be intended for reshaping, preferably straightening, keratin fibers, such as hair.

In one embodiment, the present invention may relate to a composition comprising:
(a) at least one compound selected from the group consisting of glyoxylic acid, glyoxylic acid solvates, glyoxylic acid salts, glyoxylic acid esters, glyoxylic acid amides, and mixtures thereof in an amount of more than 5% by weight, preferably more than 6% by weight, and more preferably more than 7% by weight relative to the total weight of the composition;
(b) at least one compound, different from the (a) compound, selected the group consisting of keto acids (in particular, levulinic acid), salts thereof, and mixtures thereof, in an amount of from 0.1% to 15% by weight, preferably from 0.5% to 10% by weight, and more preferably from 1% to 5% by weight, relative to the total weight of the composition, and
(c) water.

The present invention also relates to a process for reshaping, preferably straightening, keratin fibers such as hair, comprising the steps of:
(i) applying the composition according to the present invention onto the keratin fibers;
(ii) optionally rinsing off the keratin fibers with or without drying the keratin fibers after being rinsed off; and
(iii) reshaping, preferably straightening the keratin fibers, preferably at a temperature of more than 50° C., more preferably more than 100° C., and even more preferably more than 150° C.

In one embodiment, the present invention may relate to a process for reshaping, preferably straightening, keratin fibers such as hair, comprising the steps of:
(i) applying a composition onto the keratin fibers;
(ii) optionally rinsing off the keratin fibers, with or without drying the keratin fibers after being rinsed off; and
(iii) reshaping, preferably straightening, the keratin fibers preferably at a temperature of more than 50° C., more preferably more than 100° C., and even more preferably more than 150° C.,
wherein
the composition comprises
(a) at least one compound selected from the group consisting of glyoxylic acid, glyoxylic acid solvates, glyoxylic acid salts, glyoxylic acid esters, glyoxylic acid amides, and mixtures thereof in an amount of more than 5% by weight, preferably more than 6% by weight, and more preferably more than 7% by weight relative to the total weight of the composition;
(b) at least one compound, different from the (a) compound, selected the group consisting of keto acids (in particular, levulinic acid), salts thereof, and mixtures thereof, in an amount of from 0.1% to 15% by weight, preferably from 0.5% to 10% by weight, and more preferably from 1% to 5% by weight, relative to the total weight of the composition, and
(c) water.

The present invention also relates to a kit for reshaping, preferably straightening, keratin fibers such as hair, comprising:
at least one selected from the group consisting of an iron, a curler, a comb, a dryer, and a combination thereof, preferably at least one heating iron, heating curler, heating comb or heating dryer, which is capable of providing the keratin fibers with a temperature of more than 50° C., preferably more than 100° C., and more preferably more than 150° C.; and the composition according to the present invention.

### BRIEF DESCRIPTION OF DRAWINGS

FIG. 1 shows a schematic view of how to determine bending angles for Example 1 and Comparative Examples 1-4.

### BEST MODE FOR CARRYING OUT THE INVENTION

After diligent research, the inventors have found that it is possible to provide a composition which is useful for reshaping keratin fibers such as hair, based on treating the keratin fibers with glyoxylic acid or a derivative thereof, and which can provide the keratin fibers with good texture such as moisturizing feeling, strength, and resistance to humidity which can lengthen the reshaping effects.

Thus, the present invention mainly relates to a composition for keratin fibers, such as hair, comprising:
(a) at least one compound selected from the group consisting of glyoxylic acid, glyoxylic acid solvates, glyoxylic acid salts, glyoxylic acid esters, glyoxylic acid amides, and mixtures thereof; and
(b) at least one compound, different from the (a) compound, selected from the group consisting of keto acids, salts thereof, and mixtures thereof,
wherein
the amount of the (a) compound(s) in the composition is less than 15% by weight relative to the total weight of the composition, and
the amount of the (b) compound(s) in the composition is 10% by weight or less.

The present invention can provide keratin fibers such as hair with good texture, strength, and resistance to humidity which can lengthen reshaping effects.

The reshaping of keratin fibers, such as hair, here encompasses straightening or curling the keratin fibers.

The present invention can provide keratin fibers such as hair with good texture such as moisturizing feeling to touch.

The present invention can reshape or deform keratin fibers such as hair. Also, the keratin fibers which have been treated with the present invention can be bouncy.

The present invention can provide keratin fibers such as hair with resistance to humidity. Thus, for example, the keratin fibers which have been treated with the present invention to be straight can be less frizz and can show good alignment for a long period of time even under humid or wet conditions provided by, for example, rain or sweat. Accordingly, for example, the present invention can provide good hair manageability.

The present invention can provide keratin fibers such as hair with longer-lasting reshaping effects. Thus, the present invention can lengthen or prolong the reshaping effects for keratin fibers provided by using a composition including only glyoxylic acid or a derivative thereof. In particular, the present invention can further maintain the straightened or curled shape of keratin fibers, or can prevent or reduce loss of the straightened or curled shape of keratin fibers over time.

For example, if keratin fibers are originally wavy, the present invention can provide better prevention for the keratin fibers returning to the original wavy shape, after straightening the keratin fibers, in particular after shampooing.

If the composition according to the present invention comprises at least one betaine compound, the betaine compound can contribute to lengthening or prolonging the reshaping effects for the keratin fibers provided by glyoxylic acid or a derivative thereof. In particular, the use of the betaine compound can contribute to maintain the straightened or curled shape of the keratin fibers provided by glyoxylic acid or a derivative thereof, or to prevent or reduce the returning of the keratin fibers to their original shape.

The present invention can also provide keratin fibers with sufficient immediate reshaping effects. For example, the keratin fibers treated with the present invention to be straight can have less volume or less frizz due to the reduction of curls of the keratin fibers. The reshaping effects provided by the present invention can last a long time, for example, even after shampooing the keratin fibers. Thus, the present invention can also provide the shape of keratin fibers with good resistance to shampooing.

The present invention can provide keratin fibers with less damage as compared to conventional permanent reshaping processes, because the present invention does not need to use any reducing/oxidizing agents. Therefore, it can be easy to comb the keratin fibers treated by the present invention. Thus, the keratin fibers treated with the present invention can be easy to manage. Also, the keratin fibers treated by the present invention can be stronger than those treated by conventional permanent reshaping processes.

In addition, the present invention can use no or very little ammonia or thiol compound, and therefore, odor during the use of the present invention can be reduced as compared to the conventional processes which require the use of ammonia or a thiol compound. Also, the present invention can have good usability, for example, short processing time.

Hereafter, the present invention will be described in a detailed manner.

### [Composition]

The composition according to the present invention comprises:
(a) at least one compound selected from the group consisting of glyoxylic acid, glyoxylic acid solvates, glyoxylic acid salts, glyoxylic acid esters, glyoxylic acid amides, and mixtures thereof; and
(b) at least one compound, different from the (a) compound, selected from the group consisting of keto acids, salts thereof, and mixtures thereof,
wherein
the amount of the (a) compound(s) in the composition is less than 15% by weight relative to the total weight of the composition, and
the amount of the (b) compound(s) in the composition is 10% by weight or less.

It is preferable that the composition according to the present invention be a cosmetic composition, more preferably a cosmetic composition for reshaping, such as straightening, keratin fibers, and even more preferably a cosmetic composition for one-step reshaping, such as straightening, of keratin fibers. It is particularly preferable that the keratin fibers be hair.

### (Glyoxylic Acid and Derivatives Thereof)

The composition according to the present invention comprises (a) at least one compound selected from the group consisting of glyoxylic acid, glyoxylic acid solvates, glyoxylic acid salts, glyoxylic acid esters, glyoxylic acid amides, and mixtures thereof.

A single type of (a) compound may be used, or two or more different types of (a) compounds may be used in combination.

The (a) compound is selected from the group consisting of glyoxylic acid, glyoxylic acid solvates, glyoxylic acid salts, glyoxylic acid esters, glyoxylic acid amides, and mixtures thereof. In other words, the (a) compound is selected from glyoxylic acid and derivatives thereof.

As glyoxylic acid solvates, mention may be made of, for example, glyoxylic acid hydrates such as glyoxylic acid monohydrate.

As glyoxylic acid salts, mention may be made of, for example, alkaline metal salts or alkaline earth metal salts of glyoxylic acid such as sodium or potassium glyoxylate, and magnesium or calcium glyoxylate.

As glyoxylic acid esters, mention may be made of, for example, alkyl esters of glyoxylic acid such as methyl glyoxylate and ethyl glyoxylate.

As glyoxylic acid amides, mention may be made of, for example, N-glyoxyloylcarbocysteine and N-glyoxyloyl keratin amino acids.

It is preferable to use glyoxylic acid as the (a) compound.

The amount of the (a) compound(s) in the composition according to the present invention may be 0.1% by weight or more, preferably 0.5% by weight or more, and more preferably 1% by weight or more, relative to the total weight of the composition.

The amount of the (a) compound(s) in the composition according to the present invention is less than 15% by weight, preferably less than 14% by weight, and more preferably less than 13% by weight, relative to the total weight of the composition.

Thus, the amount of the (a) compound(s) in the composition according to the present invention may be from 0.1% to less than 15% by weight, preferably from 0.5% to less than 14% by weight, and more preferably from 1% to less than 13% by weight, relative to the total weight of the composition.

In one embodiment, the amount of the (a) compound(s) in the composition according to the present invention may be 12% by weight or less, preferably 11% by weight or less, and more preferably 10% by weight or less, relative to the total weight of the composition.

Thus, in this embodiment, the amount of the (a) compound(s) in the composition according to the present invention may be from 0.1% to 12% by weight, preferably from 0.5% to 11% by weight, and more preferably from 1% to 10% by weight, relative to the total weight of the composition.

In another embodiment, the amount of the (a) compound(s) in the composition according to the present invention may be more than 5% by weight, preferably more than 6% by weight, and more preferably more than 7% by weight, relative to the total weight of the composition.

Thus, in this embodiment, the amount of the (a) compound(s) in the composition according to the present invention may be more than 5% and less than 15% by weight, preferably more than 6% and less than 14% by weight, and more preferably more than 7% and less than 13% by weight, relative to the total weight of the composition, or more than 5% and 12% by weight or less, preferably more than 6% and 11% by weight or less, and more preferably more than 7% and 10% by weight or less, relative to the total weight of the composition.

### (Keto Acid)

The composition according to the present invention comprises (b) at least one compound, different from the (a) compound, selected from keto acids, salts thereof, and mixtures thereof. Two or more (b) compounds may be used in combination. Thus, a single type of (b) compound or a combination of different types of (b) compounds may be used.

The keto acids may be selected from α-keto acids, β-keto acids, γ-keto acids, and mixtures thereof.

The α-keto acids may be compounds represented by the general formula (I):

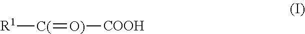

wherein
R¹ denotes a linear or branched C₁-C₆ alkyl group, optionally substituted with an OH, COOH or a halogen atom such as a chlorine or bromine atom.

The examples of α-keto acids include pyruvic acid.

The β-keto acids may be represented by the general formula (II):

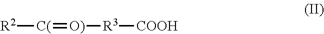

wherein
R² denotes a linear or branched C₁-C₆ alkyl group, optionally substituted with an OH, COOH or a halogen atom such as a chlorine or bromine atom; and
R³ denotes a methylene group, optionally substituted with a halogen atom such as a chlorine or bromine atom.

The examples of β-keto acids include acetoacetic acid.

The γ-keto acids may be represented by the general formula (III):

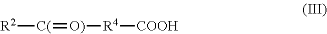

wherein
R² denotes a linear or branched C₁-C₆ alkyl group, optionally substituted with an OH, COOH or a halogen atom such as a chlorine or bromine atom; and
R⁴ denotes an ethylene group, optionally substituted with a halogen atom such as a chlorine or bromine atom.

The examples of γ-keto acids include levulinic acid.

The salts of keto acid may be alkaline metal salts or alkaline earth metal salts of keto acid such as sodium or potassium salts of keto acid, and magnesium or calcium salts of keto acid.

It is preferable to use, as the (b) compound(s), γ-keto acid, and more preferably levulinic acid and/or a salt thereof such as sodium levulinate.

The amount of the (b) compound(s) in the composition according to the present invention may be 0.1% by weight or more, preferably 0.5% by weight or more, and more preferably 1% by weight or more, relative to the total weight of the composition.

The amount of the (b) compound(s) in the composition according to the present invention is 10% by weight or less, preferably 8% by weight or less, and more preferably 6% by weight or less, relative to the total weight of the composition.

Thus, the amount of the (b) compound(s) in the composition according to the present invention may be from 0.1% to 10% by weight, preferably from 0.5% to 8% by weight, and more preferably from 1% to 6% by weight, relative to the total weight of the composition.

In one embodiment, the amount of the (b) compound(s) in the composition according to the present invention may be 5% by weight or less, preferably 4% by weight or less, and more preferably 3% by weight or less, relative to the total weight of the composition.

Thus, in this embodiment, the amount of the (b) compound(s) in the composition according to the present invention may be from 0.1% to 5% by weight, preferably from 0.5% to 4% by weight, and more preferably from 1% to 3% by weight, relative to the total weight of the composition.

In another embodiment, the amount of the (b) compound(s) in the composition according to the present invention may be from 0.5% to 10% by weight, preferably from 1% to 5% by weight, and more preferably from 2% to 4% by weight, relative to the total weight of the composition.

In another embodiment, the (b) compound(s) may be present in a lesser amount than the (a) compound(s), in the composition according to the present invention. For example, the weight ratio of the amount of the (a) compound(s) to the amount of the (b) compound(s) included in the composition according to the present invention may range from 1:1 to 15:1, preferably from 1:1 to 10:1, and more preferably from 1:1 to 4:1.

### (Water)

The composition according to the present invention may comprise (c) water.

Thus, if the composition according to the present invention comprises (c) water, the composition according to the present invention is not anhydrous.

The amount of the (c) water in the composition according to the present invention may be 20% by weight or more, preferably 30% by weight or more, and more preferably 40% by weight or more relative to the total weight of the composition.

On the other hand, the amount of the (c) water in the composition according to the present invention may be 95% by weight or less, preferably 90% by weight or less, and more preferably 85% by weight or less relative to the total weight of the composition.

The amount of (c) water in the composition according to the present invention may be from 20% to 95% by weight, preferably from 30% to 90% by weight, and more preferably from 40% to 85% by weight relative to the total weight of the composition.

### (Betaine Compound)

The composition according to the present invention may comprise (d) at least one betaine compound. A single type of (d) betaine compound may be used, or two or more different types of (d) betaine compounds may be used in combination.

The term “betaine compound” here means an amphoteric compound having a positively charged cationic moiety and a negatively charged anionic moiety, wherein no hydrogen atom is bound to a positively charged atom in the positively charged cationic moiety and the positively charged cationic moiety may not be adjacent to the negatively charged anionic moiety.

For the purpose of the present invention, the (d) betaine compound here is not a surfactant, which comprises at least one hydrophilic part and at least one hydrophobic part.

The positively charged cationic moiety in the (d) betaine compound includes, but is not limited to, a quaternary ammonium cation, a phosphonium cation, and a sulfonium cation. Preferably, the (d) betaine compound includes a quaternary ammonium cation as the positively charged cationic moiety.

The negatively charged anionic moiety in the (d) betaine compound includes, but is not limited to, a carboxylate anion.

The (d) betaine compound may be selected from the group consisting of trimethylglycine, carnitine, L-proline betaine or stachydrine, and mixtures thereof, and may preferably be trimethylglycine.

The amount of the (d) betaine compound(s) in the composition according to the present invention may be 0.01% by weight or more, preferably 0.05% by weight or more, and more preferably 0.1% by weight or more relative to the total weight of the composition.

On the other hand, the amount of the (d) betaine compound(s) in the composition according to the present invention may be 15% by weight or less, preferably 10% by weight or less, and more preferably 5% by weight or less relative to the total weight of the composition.

The amount of the (d) betaine compound(s) in the composition according to the present invention may range from 0.01% to 15% by weight, preferably from 0.05% to 10% by weight, and more preferably from 0.1% to 5% by weight relative to the total weight of the composition.

(pH)

The composition according to the present invention may have a pH of 7 or less, preferably 6 or less, and more preferably 5 or less, which is measured at 25° C.

The composition according to the present invention may have a pH of 3 or more, which is measured at 25° C.

The composition according to the present invention may have a pH of from 3 to 7, preferably from 3 to 6, and more preferably from 3 to 5, which is measured at 25° C.

### (Alkaline Agent)

The composition according to the present invention, may comprise at least one alkaline agent. Two or more alkaline agents may be used in combination. Thus, a single type of alkaline agent or a combination of different types of alkaline agents may be used.

The alkaline agent is different from the (d) betaine compound.

It is preferable that the composition according to the present invention include alkaline agent(s), if the (a) compound or the compound (b) can function to reduce the pH of the composition.

The alkaline agent may be an inorganic alkaline agent. It is preferable that the alkaline agent be non-volatile. It is preferable that the inorganic alkaline agent be selected from the group consisting of alkaline metal hydroxides; alkaline earth metal hydroxides; and alkaline metal phosphates and monohydrogen phosphates such as sodium phosphate or sodium monohydrogen phosphate.

As examples of the inorganic alkaline metal hydroxides, mention may be made of sodium hydroxide, lithium hydroxide and potassium hydroxide. As examples of the alkaline earth metal hydroxides, mention may be made of calcium hydroxide and magnesium hydroxide. As the inorganic alkaline agent, sodium hydroxide and potassium hydroxide are preferable.

The alkaline agent may be an organic alkaline agent. It is preferable that the organic alkaline agent be selected from the group consisting of monoamines and diamines.

As examples of the monoamines, mention may be made of alkanolamines such as mono-, di- and tri-ethanolamine, comprising 1 to 3 hydroxyalkyl (C₁-C₄) groups. Particularly, alkanolamines may be selected from monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, and tris(hydroxymethylamino)methane.

The diamines may be described by the structure (B) below:

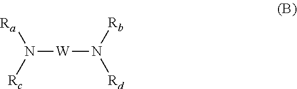

wherein W denotes an alkylene such as propylene optionally substituted by a hydroxyl or a C₁-C₄ alkyl radical, and Rₐ, R_{b}, R_{c} and R_{d} independently denote a hydrogen atom, an alkyl radical or a C₁-C₄ hydroxyalkyl radical, which may be exemplified by 1,3-propanediamine and derivatives thereof.

The amount of the alkaline agent(s) in the composition according to the present invention may be 0.01% by weight or more, preferably 0.05% by weight or more, and more preferably 0.1% by weight or more, relative to the total weight of the composition.

On the other hand, the amount of the alkaline agent(s) in the composition according to the present invention may be 15% by weight or less, preferably 10% by weight or less, and more preferably 5% by weight or less, relative to the total weight of the composition.

Thus, the amount of the alkaline agent(s) in the composition according to the present invention may be from 0.01% to 15% by weight, preferably from 0.05% to 10% by weight, and more preferably from 0.1% to 5% by weight, relative to the total weight of the composition.

### (Acid)

The composition according to the present invention may comprise at least one acid which is different from the (a) compound or the (b) compound (as well as the (d) betaine compound, if present). Two or more acids may be used in combination. Thus, a single type of acid or a combination of different types of acids may be used.

The acid may be used to adjust the pH of the composition according to the present invention.

As the acid, mention may be made of any inorganic or organic acids which are commonly used in cosmetic products such as citric acid, lactic acid, sulfuric acid, phosphoric acid or hydrochloric acid (HCl). HCl is preferable.

The amount of the acid(s) in the composition according to the present invention may be 0.01% by weight or more, preferably 0.05% by weight or more, and more preferably 0.1% by weight or more, relative to the total weight of the composition.

On the other hand, the amount of the acid(s) in the composition according to the present invention may be 15% by weight or less, preferably 10% by weight or less, and more preferably 5% by weight or less, relative to the total weight of the composition.

Thus, the amount of the acid(s) in the composition according to the present invention may be from 0.01% to 15% by weight, preferably from 0.05% to 10% by weight, and more preferably from 0.1% to 5% by weight, relative to the total weight of the composition.

### (Other Optional Ingredients)

The composition according to the present invention may also comprise at least one other optional ingredient, chosen in particular from: oils, preferably polar oils, and more preferably ester oils, in the form of a liquid at 25° C. under atmospheric pressure; solid fatty substances and in particular C₈-C₄₀ esters, C₈-C₄₀ acids and C₈-C₄₀ alcohols, at 25° C. under atmospheric pressure; thickeners; sunscreens; moisturizers; antidandruff agents; antioxidants; antibacterial agents such as benzoic acid; preservatives such as phenoxyethanol; chelating agents; nacreous agents and opacifiers; plasticizers or coalescers; fillers; emulsifiers; polymers, in particular conditioning polymers, such as cationic polymers; fragrances; silanes; crosslinking agents; and surfactants including anionic, amphoteric and nonionic surfactants. The composition can, of course, comprise several cosmetic ingredients appearing in the above list.

The composition according to the present invention may include one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol, and caprylyl glycol; other polyols such as glycerol; and ethers such as ethylene glycol monomethyl, monoethyl, and monobutyl ethers, propylene glycol monomethyl, monoethyl, and monobutyl ethers, and butylene glycol monomethyl, monoethyl, and monobutyl ethers.

Depending on their nature and the purpose of the composition, the above optional ingredient(s) can be present in normal amounts which can be easily determined by those skilled in the art and which can be, for each ingredient, between 0.01% and 80% by weight. Those skilled in the art will take care to choose the ingredients included in the composition, and also the amounts thereof, such that they do not harm the properties of the compositions used for the present invention.

### (Ammonia and Thiol Compound)

It is preferable that the composition according to the present invention be free of ammonia or thiol compound. The term “free of ammonia or thiol compound” means that the composition according to the present invention does not include a substantial amount of ammonia or thiol compound. Preferably the composition according to the present invention includes 1% by weight or less, more preferably 0.5% by weight or less, and even more preferably 0.1% by weight or less of ammonia or thiol compound, in particular no ammonia or thiol compound.

Due to the very small amount or the absence of ammonia and/or thiol compound, malodor during the use of the composition according to the present invention can be reduced or prevented.

The thiol compound here means a compound which has at least one thiol (—SH) group.

The thiol compound may be a reducing agent. The thiol reducing agent may be chosen from the group consisting of thioglycolic acid and derivatives thereof, in particular esters thereof such as glycerol or glycol monothioglycolate; thiolactic acid and derivatives thereof, in particular esters thereof such as glycerol monothiolactate; 3-mercaptopropionic acid and derivatives thereof, in particular esters thereof such as glycerol 3-mercaptopropionate and ethyleneglycol 3-mercaptopropionate; cysteamine and derivatives thereof, in particular C₁-C₄ acyl derivatives thereof such as N-acetylcysteamine and N-propionylcysteamine; mono-thioglycerol and derivatives thereof, in particular esters; cysteine and derivatives thereof, in particular esters such as N-acetylcysteine, N-alkanoylcysteine and cysteine alkyl esters; thioglycerine and derivatives thereof, in particular s-alkyl derivatives; and salts thereof.

As the above salts, mention may be made of, for example, ammonium salts; primary-, secondary- or tertiary-amine salts; alkaline metal salts; and alkaline earth metal salts. As the primary-, secondary- or tertiary-amine, for example, monoethanolamine, di-isopropanolamine or triethanolamine, respectively, may be mentioned.

Other examples of the thiol reducing agent include, but are not limited to, sugar N-mercapto alkyl amides such as N-(mercapto-2-ethyl) gluconamide, β-mercaptopropionic acid and derivatives thereof; thiomalic acid; pantheteine; N-(mercaptoalkyl) @-hydroxyalkyl amides such as those described in European Patent Application No. 0 354 835 and N-mono- or N,N-dialkylmercapto 4-butyramides such as those described in European Patent Application No. 0 368 763; aminomercaptoalkyl amides such as those described in European Patent Application No. 0 432 000 and alkylaminomercaptoalkylamides such as those described in European Patent Application No. 0 514 282; (2/3) hydroxy-2 propyl thioglycolate; and the hydroxy-2 methyl-1 ethyl thioglycolate-based mixture (67/33) described in French Patent Application No. 2 679 448.

### (Reducing Agent and Oxidizing Agent)

The composition according to the present invention may comprise a reducing agent; however, it is preferable that the composition according to the present invention comprises a reduced amount of a reducing agent or an oxidizing agent, preferably free of a reducing agent or an oxidizing agent.

The term “free of a reducing agent or an oxidizing agent” means that the composition according to the present invention does not include a substantial amount of a reducing agent or an oxidizing agent. Preferably the composition according to the present invention includes 1% by weight or less, more preferably 0.5% by weight or less, and even more preferably 0.1% by weight or less of a reducing agent or an oxidizing agent, in particular no reducing agent or no oxidizing agent.

The reducing agent may be a thiol reducing agent or a non-thiol reducing agent. The thiol reducing agent is as described above.

The non-thiol reducing agent here means a reducing agent with no thiol group. The non-thiol reducing agent may be chosen from the group consisting of sulfites, bisulfites, sulfinates, phosphines, sugars, reductones and hydrides. The non-thiol reducing agent may be selected from ammonium sulfites and bisulfites as well as metal sulfites and bisulfites, more preferably alkali metal or alkali earth metal sulfites and bisulfites, and more preferably sodium sulfites and bisulfites.

The oxidizing agent may be chosen from hydrogen peroxide, alkali metal bromates, ferricyanides peroxygenated salts, and compounds capable of producing hydrogen peroxide by hydrolysis. For example, the oxidizing agent can be chosen from aqueous hydrogen peroxide solution, urea peroxide, alkali metal bromates and persalts such as perborates and persulphates.

### (Form)

The compositions used for the present invention may be in any form suitable for topical application, and in particular in the form of an aqueous, alcoholic or aqueous-alcoholic, or oily solution or suspension; a solution or a dispersion of a lotion or serum type; an emulsion, in particular of liquid or semi-liquid consistency, of O/W, W/O or multiple type (if the composition according to the present invention includes at least one oil); a suspension or emulsion of a soft consistency of cream (O/W) or (W/O) type; an aqueous gel; or any other cosmetic form.

The compositions used for the present invention may be in any galenical forms. For example, the compositions used for the present invention may be in the form of any treatment for hair in general, such as a cream, a lotion, a gel, and the like.

The composition according to the present invention can be used for treating, preferably reshaping, and more preferably straightening keratin fibers such as hair.

### [Process]

The present invention also relates to a process for reshaping, preferably straightening, keratin fibers, such as hair, comprising the steps of:
(i) applying the composition according to the present invention onto the keratin fibers;
(ii) optionally rinsing off the keratin fibers with or without drying the keratin fibers after being rinsed off; and
(iii) reshaping, preferably straightening, the keratin fibers, preferably at a temperature of more than 50° C., more preferably more than 100° C., and even more preferably more than 150° C.

The details of the composition used in the process according to the present invention are explained in the section titled [Composition] above.

The process according to the present invention is for the purpose of reshaping, preferably straightening, keratin fibers such as hair, more preferably straightening for a long period of time, and even more preferably straightening even after shampooing several times. In this sense, the process according to the present invention can be a semi-permanent reshaping, preferably straightening, process.

It is preferable that the keratin fibers be cleaned before the step (i). This cleaning step may be performed by, for example, shampooing the keratin fibers, followed by rinsing off the keratin fibers. After rinsing off, the keratin fibers may be dried. It is preferable that the keratin fibers are wet just before the step (i).

In the step (i), the composition according to the present invention, which is explained above, is applied to keratin fibers such as hair. The application of the composition according to the present invention may be performed by any means, such as a brush or a comb.

The bath ratio of the applied composition according to the present invention to the keratin fibers may range from 0.1 to 10, more particularly from 0.5 to 5, and preferably between 0.3 and 2. The term “bath ratio” is intended to mean the weight ratio between the total weight of the applied composition and the total weight of the keratin fibers.

It may be possible that, after the application of the composition according to the present invention, the keratin fibers be left as they are for a certain amount of time; typically from 1 minute to 1 hour, preferably from 2 to 30 minutes, and more preferably from 3 to 10 minutes, if necessary, in order to let the composition penetrate into the keratin fibers.

After the step (i), the process according to the present invention may comprise the optional step (ii) of rinsing off the keratin fibers with or without drying the keratin fibers after being rinsed off.

Thus, if the step (ii) is present, the keratin fibers are rinsed off in order to remove the composition according to the present invention from the keratin fibers. The step of rinsing may be performed with water. It is preferable that the keratin fibers be dried after being rinsed off. The drying of the keratin fibers can be performed with a conventional drying means such as a hair dryer.

On the other hand, if the step (ii) is not present, the keratin fibers can be treated by the step (iii) after the step (i) without being rinsed off.

In the step (iii), the keratin fibers, which are preferably dry, are subjected to reshaping, preferably straightening, preferably at a temperature of more than 50° C., more preferably more than 100° C., and even more preferably more than 150° C., with a heater, such as a heating iron and a heating curler. This temperature may be less than 250° C., preferably less than 240° C., and more preferably less than 230° C. Thus, this temperature may be more than 50° C. and less than 250° C., preferably more than 100° C. and less than 240° C., and more preferably more than 150° C. and less than 230° C.

The reshaping may be performed by providing the keratin fibers with any mechanical force such as mechanical tension. The mechanical force can be applied to the keratin fibers by any reshaping means to deform the keratin fibers to an intended shape. For example, the mechanical power may be provided by at least one reshaping means selected from the group consisting of at least one iron, at least one curler, at least one comb, and a combination thereof. Any conventional iron, curler or comb may be used as the reshaping means. A dryer which can blow gas such as air may also be used as a reshaping means.

The reshaping means may comprise at least one heater. Thus, the reshaping means may be at least one heating iron and/or at least one heating curler and/or at least one heating comb and/or at least one heating dryer which can blow hot gas such as hot air. It may be preferable to use at least one heating iron and/or at least one heating curler.

As the heating iron, any conventional heating iron can be used. The heating iron can have at least one plate, preferably two plates, which can be heated by, for example, electric heating. The heated plate(s) can be applied onto the keratin fibers and moved along the direction of the keratin fibers to straighten them.

It is preferable that the heating iron has two plates, and that keratin fibers are sandwiched between the two plates of the heating iron, in which at least one of the plates can be heated, and then the two plates are moved along the direction of the keratin fibers to straighten them.

It is preferable that the heating iron, in particular a part thereof contacting with keratin fibers, such as a heating plate or heating plates, has a temperature of more than 50° C. and less than 250° C., more preferably more than 100° C. and less than 240° C., and even more preferably more than 150° C. and less than 230° C.

The step (iii) can be performed by one or more strokes of moving the heating iron along the direction of the keratin fibers. The step (iii) can be controlled by not only the temperature of the heating iron but also the number of strokes of the heating iron.

The heating time may depend on the temperature of the heating iron but also on the number of strokes of the heating iron. It may be, for example, from 1 second to 10 minutes, and preferably from a few seconds to 5 minutes.

As the heating curler, any conventional heating curler can be used. If the keratin fibers are rolled around a heating curler, this rolling-up may be performed on the entire length of the keratin fibers or, for example, on half the length of the keratin fibers. Depending on, for example, the desired hairstyle shape and amount of curls, the rolling-up may be performed with more or less thick locks.

It is preferable that the heating curler, in particular a part thereof contacting with keratin fibers, such as a heating rod and a heating cover, has a temperature of more than 50° C. and less than 250° C., more preferably more than 100° C. and less than 240° C., and even more preferably more than 150° C. and less than 230° C.

The step (iii) can be performed by maintaining keratin fibers on a heating curler such that mechanical tension is applied on the keratin fibers. The step (iii) can be controlled by not only the temperature of the heating curler but also the strength of the mechanical tension.

The heating time may depend on the temperature of the heating curler but also on the strength of the mechanical tension. It may be, for example, from 1 second to 10 minutes, and preferably from a few seconds to 5 minutes.

If necessary, before the step (ii), mechanical tension may be applied to keratin fibers as explained above.

### [Kit]

The present invention also relates to a kit for reshaping, preferably straightening, keratin fibers such as hair, comprising:

at least one selected from the group consisting of an iron, a curler, a comb, a dryer, and a combination thereof, preferably at least one heating iron, heating curler, heating comb or heating dryer which is capable of providing the keratin fibers with a temperature of more than 50° C., preferably more than 100° C., and more preferably more than 150° C.; and the composition according to the present invention.

Any conventional iron or curler, including any conventional heating iron or heating curler, may be used as the above iron or curler in the kit according to the present invention.

The heating iron or heating curler in the kit according to the present invention is not limited as long as it can heat keratin fibers at a temperature of more than 50° C., preferably more than 100° C., and more preferably more than 150° C. This temperature may be less than 250° C., preferably less than 240° C., and more preferably less than 230° C. Thus, this temperature may be more than 50° C. and less than 250° C., preferably more than 100° C. and less than 240° C., and more preferably more than 150° C. and less than 230° C.

The iron or curler, as well as the composition, in the kit according to the present invention may be used as explained in the section titled [Process] above.

The details of the composition used in the kit according to the present invention are explained in the section titled [Composition] above.

### [Use]

The present invention may relate to a use of
(a) at least one compound selected from the group consisting of glyoxylic acid, glyoxylic acid solvates, glyoxylic acid salts, glyoxylic acid esters, glyoxylic acid amides, and mixtures thereof; and
(b) at least one compound, different from the (a) compound, selected from the group consisting of keto acids, salts thereof, and mixtures thereof,
in a composition for reshaping, preferably straightening, keratin fibers such as hair, preferably with heating, wherein
the amount of the (a) compound(s) in the composition is less than 15% by weight relative to the total weight of the composition, and
the amount of the (b) compound(s) in the composition is 10% by weight or less,
in order to provide keratin fibers such as hair treated by the composition with good texture, strength, and resistance to humidity,
and/or
in order to lengthen the reshaping effects for keratin fibers treated by the composition, in particular in order to further maintain the straight shape of the keratin fibers or to prevent or reduce waving of the keratin fibers.

The heating is not limited as long as it can heat keratin fibers at a temperature of more than 50° C., preferably more than 100° C., and more preferably more than 150° C. This temperature may be less than 250° C., preferably less than 240° C., and more preferably less than 230° C. Thus, this temperature may be more than 50° C. and less than 250° C., preferably more than 100° C. and less than 240° C., and more preferably more than 150° C. and less than 230° C.

The above composition may further comprise (c) water and/or (d) at least one betaine compound.

The above explanations regarding the (a) compound, the (b) compound, the (c) water, and the (d) betaine compound can apply to those in the above composition used for the use according to the present invention.

### EXAMPLES

The present invention will be described in a more detailed manner by way of examples. However, these examples should not be construed as limiting the scope of the present invention.

### Example 1 and Comparative Examples 1-4

### [Preparation]

Each of the compositions according to Example 1 and Comparative Examples 1-4 was prepared by mixing the ingredients shown in Table 1. The numerical values for the amounts of the ingredients are all based on “% by weight” as raw materials.

TABLE 1

| | | Comp. | Comp. | Comp. | Comp. |
|---|---|---|---|---|---|
| | Ex. 1 | Ex. 1 | Ex. 2 | Ex. 3 | Ex. 4 |
| Dipropylene Glycol | 10.1 | 10.1 | 10.1 | 10.1 | 10.1 |
| Hydroxyethylcellulose | 0.7 | 0.7 | 0.7 | 0.7 | 0.7 |
| Phenoxyethanol | 0.8 | 0.8 | 0.8 | 0.8 | 0.8 |
| Glyoxylic Acid (50 wt | 14 | — | 14 | 30 | 14 |
| % AM) (1) | | | | | |
| Levulinic Acid (60 wt | 4 | — | — | 4 | 18.2 |
| % AM) (2) | | | | | |
| pH adjuster | qs pH 3 | qs pH 3 | qs pH 3 | qs pH 3 | qs pH 3 |
| Water | qsp 100 | qsp 100 | qsp 100 | qsp 100 | qsp 100 |
| Texture | 4 | 3 | 3 | 3 | 3 |
| Bending Resistance (°) | 74.3 | 62.4 | 67.3 | 65.5 | 57.6 |

AM: Active Material
(1) sold by the company WEYLCHEM under the name GLYOXO-HIGH PURE 50 (containing 50% active material).
(2) sold by the company DR STRAETMANS (EVONIK) under the name DERMOSOFT 700 B (containing 60% active material).

### [Evaluations]

### (Texture)

Each of the compositions according to Example 1 and Comparative Examples 1-4 was used to treat a hair swatch (1.0 g, 27 cm) in accordance with the following steps.
1. The hair swatch was shampooed and rinsed with water.
2. After rinsing, 1 g of the composition to be evaluated was applied onto the wet hair swatch within 1 minute.
3. The hair swatch was left for 10 minutes at room temperature (25° C.).
4. The hair swatch was rinsed off thoroughly with tap water.
5. The hair swatch was dried with a blow dryer.

Just after drying the hair swatch, “moisturizing feeling” of the hair swatch was evaluated by a professional panelist, relative to the benchmark (treated with the composition according to Comparative Example 1) based on the following criteria. The higher the score is, the better the moisturizing feeling of the hair is.
5: Very good
4: Good
3: Parity to benchmark (hair swatch treated with water: Comparative Example 1)
2: Poor
1: Very poor

The results are shown in the row “Texture” in Table 1.

It is clear that the composition according to Example 1 provided better texture (better moisturizing feeling) than the compositions according to Comparative Examples 1-4.

Comparative Examples 1-4 show that the use of only glyoxylic acid (Comp. Ex. 2), the use of 15% by weight or more of glyoxylic acid (Comp. Ex. 3) or the use of more than 10% by weight of levulinic acid (18.2*0.6=10.92 wt %) could not improve the texture of the hair swatch.

### (Bending Resistance)

Each of the compositions according to Example 1 and Comparative Examples 1-4 was used to treat a hair swatch (1.0 g, 27 cm) in accordance with the following steps.
1. The hair swatch was shampooed and rinsed with water.
2. After rinsing, 1 g of the composition to be evaluated was applied onto the wet hair swatch within 1 minute.
3. The hair swatch was left for 10 minutes at room temperature (25° C.).
4. The hair swatch was rinsed off thoroughly with tap water.
5. The hair swatch was dried with a blow dryer.

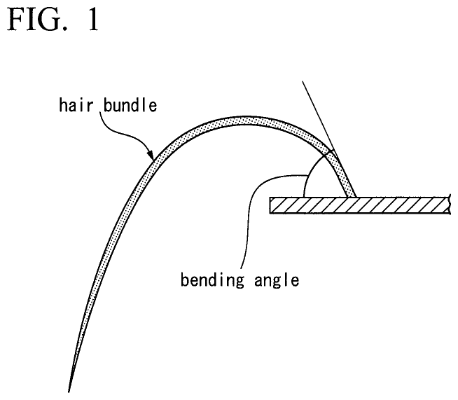

The root of the hair swatch was fixed on a plate. On the other hand, the tip of the hair swatch was held and oriented upwardly, and then, the tip of the hair swatch was released such that the tip of the hair swatch could be directed downwardly. The angle between the plate and the root of the hair swatch (hair bundle) was measured (cf. FIG. 1). The average value was determined as the bending angle.

The bending angles are shown in the row “Bending Resistance” in Table 1. The higher the bending angle is, the bouncier the hair is.

It is clear that the composition according to Example 1 provided more bending resistance (more strength) than the compositions according to Comparative Examples 1-4.

Comparative Examples 1-4 show that the use of only glyoxylic acid (Comp. Ex. 2), the use of 15% by weight or more of glyoxylic acid (Comp. Ex. 3) or the use of more than 10% by weight of levulinic acid (18.2*0.6=10.92 wt %) could not increase sufficiently the strength of the hair swatch.

### Example 2 and Comparative Examples 5-8

### [Preparation]

Each of the compositions according to Example 2 and Comparative Examples 5-8 was prepared by mixing the ingredients shown in Table 2. The numerical values for the amounts of the ingredients are all based on “% by weight” as raw materials.

TABLE 2

| | | Comp. | Comp. | Comp. | Comp. |
|---|---|---|---|---|---|
| | Ex. 2 | Ex. 5 | Ex. 6 | Ex. 7 | Ex. 8 |
| Isopropyl Myristate | 10 | — | 10 | 10 | 10 |
| Glycerin | 4 | — | 4 | 4 | 4 |
| Cetearyl Alcohol | 4 | — | 4 | 4 | 4 |
| Steareth-2 | 0.5 | — | 0.5 | 0.5 | 0.5 |
| Steareth-10 | 0.5 | — | 0.5 | 0.5 | 0.5 |
| Stearamidopropyl | 2.5 | — | 2.5 | 2.5 | 2.5 |
| Dimethylamine | | | | | |
| Water | qsp 100 | qsp 100 | qsp 100 | qsp 100 | qsp 100 |
| Glyoxylic Acid (50 wt | 20 | — | — | 20 | — |
| % AM) (1) | | | | | |
| Levulinic Acid (60 wt | 5 | — | — | — | 5 |
| % AM) (2) | | | | | |
| Betaine | 1 | — | 1 | 1 | 1 |
| pH adjuster | qs pH 3 | — | qs pH 3 | qs pH 3 | qs pH 3 |
| Ethanol | 3 | — | 3 | 3 | 3 |
| Shape Transformation | 5 | 1 | 1 | 2 | 3 |
| Speed | | | | | |
| Frizzness | 4 | 1 | 1 | 3 | 3 |
| Alignment | 4 | 1 | 1 | 3 | 2 |

AM: Active Material
(1) sold by the company WEYLCHEM under the name GLYOXO-HIGH PURE 50 (containing 50% active material).
(2) sold by the company DR STRAETMANS (EVONIK) under the name DERMOSOFT 700 B (containing 60% active material).

### [Evaluations]

### (Shape Transformation Speed)

Each of the compositions according to Example 2 and Comparative Examples 5-8 was used to treat a hair swatch (1.0 g, 27 cm) in accordance with the following steps.
1. The hair swatch was shampooed and rinsed with water.
2. After rinsing, 0.6 g of the composition to be evaluated was applied onto the wet hair swatch within 1 minute.
3. The hair swatch was left for 10 minutes at room temperature (25° C.).
4. The hair swatch was rinsed off thoroughly with tap water.
5. The hair swatch was dried with a blow dryer.
6. The hair swatch was subjected to straightening by 3 strokes with a hair straightening iron (ADST Premium DS2, Hakko Limited, Japan) at 180° C. for 4 seconds/stroke.
7. The hair swatch was put in an oven at 50° C. for 30 minutes to be standardized.
8. The hair swatch was placed in a controlled chamber (ETS Environmental Test Chamber, Model 5532-1-30-2262) at a temperature of 30° C. and under an atmospheric humidity of 70% for 1 hour.
9. A video was taken of the hair swatch during step 8.

The speed of shape transformation of the hair swatch was evaluated by the video taken in step 9 in accordance with the following criteria. The higher the score is, the better the resistance to humidity is.
5: Slow
4: Somewhat Slow
3: Normal
2: Somewhat Fast
1. Fast

The results are shown in the row “Shape Transformation Speed” in Table 2.

It is clear that the composition according to Example 2 provided slower speed of shape transformation (i.e., it takes more time for the hair to change its shape) than the compositions according to Comparative Examples 5-8. This means that the composition according to Example 2 provided more resistance to humidity than the compositions according to Comparative Examples 5-8.

In particular, Comparative Examples 7 and 8 show that the use of only glyoxylic acid (Comp. Ex. 7) or the use of only levulinic acid/sodium levulinate could not increase sufficiently the resistance to humidity as the composition according to Example 2 did.

### (Frizzness)

Each of the compositions according to Example 2 and Comparative Examples 5-8 was used to treat a hair swatch (1.0 g, 27 cm) in accordance with the following steps.
1. The hair swatch was shampooed and rinsed with water.
2. After rinsing, 0.6 g of the composition to be evaluated was applied onto the wet hair swatch within 1 minute.
3. The hair swatch was left for 10 minutes at room temperature (25° C.).
4. The hair swatch was rinsed off thoroughly with tap water.
5. The hair swatch was dried with a blow dryer.
6. The hair swatch was subjected to straightening by 3 strokes with a hair straightening iron (ADST Premium DS2, Hakko Limited, Japan) at 180° C. for 4 seconds/stroke.
7. The hair swatch was put in an oven at 50° C. for 30 minutes to be standardized.
8. A photograph of the hair swatch was taken just after step 7.
9. The hair swatch was placed in a controlled chamber (ETS Environmental Test Chamber, Model 5532-1-30-2262) at a temperature of 30° C. and under an atmospheric humidity of 70% for 1 hour.
10. A photograph of the hair swatch was taken just after step 9.

The frizzness of the hair swatch was evaluated in accordance with the following criteria by comparing the photographs taken in steps 8 and 10. The higher the score is, the better the resistance to humidity is.
5: Very Good (no frizzes)
4: Good (little frizzes)
3: Fair (few frizzes)
2: Poor (some frizzes)
1: Very Poor (many frizzes)

The results are shown in the row “Frizzness” in Table 2.

It is clear that the composition according to Example 2 provided less frizzes under humid conditions than the compositions according to Comparative Examples 5-8. This means that the composition according to Example 2 provided more resistance to humidity than the compositions according to Comparative Examples 5-8.

In particular, Comparative Examples 7 and 8 show that the use of only glyoxylic acid (Comp. Ex. 7) or the use of only levulinic acid/sodium levulinate could not suppress frizzness as the composition according to Example 2 did.

### (Alignment)

Each of the compositions according to Example 2 and Comparative Examples 5-8 was used to treat a hair swatch (1.0 g, 27 cm) in accordance with the following steps.
1. The hair swatch was shampooed and rinsed with water.
2. After rinsing, 0.6 g of the composition to be evaluated was applied onto the wet hair swatch within 1 minute.
3. The hair swatch was left for 10 minutes at room temperature (25° C.).
4. The hair swatch was rinsed off thoroughly with tap water.
5. The hair swatch was dried with a blow dryer.
6. The hair swatch was subjected to straightening by 3 strokes with a hair straightening iron (ADST Premium DS2, Hakko Limited, Japan) at 180° C. for 4 seconds/stroke.
7. The hair swatch was put in an oven at 50° C. for 30 minutes to be standardized.
8. A photograph of the hair swatch was taken just after step 7.
9. The hair swatch was placed in a controlled chamber (ETS Environmental Test Chamber, Model 5532-1-30-2262) at a temperature of 30° C. and under an atmospheric humidity of 70% for 1 hour.
10. A photograph of the hair swatch was taken just after step 9.

The alignment of the hair swatch was evaluated in accordance with the following criteria by comparing the photographs taken in steps 8 and 10. The higher the score is, the better the resistance to humidity is.
5: Very good
4: Good
3: Fair
2: Poor
1: Very Poor

The results are shown in the row “Alignment” in Table 2.

It is clear that the composition according to Example 2 provided better alignment of hair under humid conditions than the compositions according to Comparative Examples 5-8. This means that the composition according to Example 2 provided more resistance to humidity than the compositions according to Comparative Examples 5-8.

In particular, Comparative Examples 7 and 8 show that the use of only glyoxylic acid (Comp. Ex. 7) or the use of only levulinic acid/sodium levulinate could not provide good alignment of hair as the composition according to Example 2 did.

1-15. (canceled)

16. A composition for reshaping keratin fibers, comprising:
(a) at least one compound chosen from glyoxylic acid, glyoxylic acid solvates, glyoxylic acid salts, glyoxylic acid esters, glyoxylic acid amides, or mixtures thereof; and
(b) at least one compound, different from the at least one compound (a), chosen from keto acids, salts thereof, or mixtures thereof,
wherein:
the total amount of the at least one compound (a) is less than 15% by weight, relative to the total weight of the composition, and
the total amount of the at least one compound (b) in the composition is equal to or less than 10% by weight, relative to the total weight of the composition.

17. The composition of claim 16, wherein the total amount of the at least one compound (a) is at least 0.1% by weight, relative to the total weight of the composition.

18. The composition of claim 16, wherein the total amount of the at least one compound (a) is at least 1% by weight, relative to the total weight of the composition.

19. The composition of claim 16, wherein the at least one compound (b) is chosen from levulinic acid, a salt thereof, or a mixture thereof.

20. The composition of claim 16, wherein the total amount of the at least one compound (b) is at least 0.1% by weight, relative to the total weight of the composition.

21. The composition of claim 16, wherein the total amount of the at least one compound (b) is at least 1% by weight, relative to the total weight of the composition.

22. The composition of claim 16, wherein the composition further comprises (c) water.

23. The composition of claim 16, wherein the total amount of water (c) ranges from 20% to 95% by weight, relative to the total weight of the composition.

24. The composition of claim 22, wherein the total amount of water (c) ranges from 40% to 85% by weight, relative to the total weight of the composition.

25. The composition of claim 16, wherein the composition further comprises (d) at least one betaine compound.

26. The composition of claim 25, wherein the at least one betaine compound (d) is chosen from trimethylglycine, carnitine, L-proline betaine, stachydrine, or mixtures thereof.

27. The composition of claim 25, wherein the total amount of the at least one betaine compound (d) ranges from 0.01% to 15% by weight, relative to the total weight of the composition.

28. The composition of claim 16, wherein the pH of the composition is 7 or less.

29. The composition of claim 16, wherein the composition does not comprise an ammonia compound or a thiol compound.

30. The composition of claim 16, wherein the composition comprises an ammonia compound in a total amount of less than 1% by weight, or comprises a thiol compound in a total amount of less than 1% by weight, relative to the total weight of the composition.

31. The composition of claim 16, wherein the composition does not comprise a reducing agent or an oxidizing agent.

32. The composition of claim 16, wherein the composition comprises a reducing agent in a total amount of less than 1% by weight, or comprises an oxidizing agent in a total amount of less than 1% by weight, relative to the total weight of the composition.

33. The composition of claim 16, wherein the composition is a composition for straightening keratin fibers.

34. A method for straightening keratin fibers, comprising:
(i) applying to the keratin fibers a composition comprising:
(a) at least one compound chosen from glyoxylic acid, glyoxylic acid solvates, glyoxylic acid salts, glyoxylic acid esters, glyoxylic acid amides, or mixtures thereof; and
(b) at least one compound, different from the at least one compound (a), chosen from keto acids, salts thereof, or mixtures thereof,
wherein:
the amount of the at least one compound (a) in the composition is less than 15% by weight, relative to the total weight of the composition, and
the amount of the at least one compound (b) in the composition is equal to or less than 10% by weight, relative to the total weight of the composition;
(ii) rinsing off the keratin fibers;
(iia) optionally drying the keratin fibers after rinsing; and
(iii) straightening the keratin fibers at a temperature of greater than 50° C.

35. A kit for reshaping keratin fibers, comprising:
(i) at least one means for reshaping keratin fibers chosen from an iron, a curler, a comb, a dryer, or a combination thereof, configured to heat the keratin fibers at a temperature of greater than 50° C.; and
(ii) a composition comprising:
(a) at least one compound chosen from glyoxylic acid, glyoxylic acid solvates, glyoxylic acid salts, glyoxylic acid esters, glyoxylic acid amides, or mixtures thereof; and
(b) at least one compound, different from the at least one compound (a), chosen from keto acids, salts thereof, or mixtures thereof,
wherein:
the total amount of the at least one compound (a) in the composition is less than 15% by weight, relative to the total weight of the composition, and
the total amount of the at least one compound (b) in the composition is equal to or less than 10% by weight, relative to the total weight of the composition.
